(19) United States
(12) Patent Application Publication
(10) Pub. No.: US 2006/0204952 A1
(43) Pub. Date: Sep. 14, 2006

(54) Technique for measuring cell-membrane properties in cultured cells grown on biomaterials in an Ussing chamber

(75) Inventors: Alan Moy, Iowa City, IA (US); Sunaina Fotedar, Columbia, MD (US); Anthony English, Knoxville, TN (US)

Correspondence Address: FULBRIGHT & JAWORSKI L.L.P., 600 CONGRESS AVE., SUITE 2400, AUSTIN, TX, 78701, US

(73) Assignee: Iowa Research Foundation

(21) Appl. No.: 11/357,444

(22) Filed: Feb. 17, 2006

Related U.S. Application Data
(60) Provisional application No. 60/654,121, filed on Feb. 18, 2005.

(51) Int. Cl. C12Q 1/00; C12N 5/06

(52) U.S. Cl. 435/4; 435/325; 205/777

(57) ABSTRACT

The present invention is directed to a system and method which measures transcellular parameters in which cells are cultured in a monolayer on a biomaterial. The parameters measured are indicative of in vivo values such as cell-cell and cell-matrix adhesion parameters.

The instant application claims benefit of priority to U.S. Provisional Application Ser. No. 60/654,121, filed Feb. 18, 2005, the entire contents of which are hereby incorporated by reference.

### STATEMENT REGARDING FEDERALLY SPONSORED RESEARCH OR DEVELOPMENT

This invention was made in part with government support under Grant No. GM61732 awarded by the National Institutes of Health and Grant No. BES-0238905 awarded by National Science Foundation. The United States Government may have certain rights in the invention.

### TECHNICAL FIELD

The present invention relates to the field of cell biology. More specifically, the present invention utilizes a method and system to measure transcellular parameters of culture cells using a modified Ussing chamber system.

### BACKGROUND OF THE INVENTION

Endothelial cells play a crucial role in maintaining homeostasis (Silvia et al., 2002). They perform a large array of physiological functions that are influenced by their cellular homogeneity in the different vascular beds (Chand et al., 1981). They are vital for the proper functioning of blood vessels wherein a monolayer of these endothelial cells lines the blood vessels and helps regulate blood flow, inhibits smooth muscle cell overgrowth, and resists thrombosis (Liu et al., 2002). The endothelium lining the inner wall of blood vessel is a complex physiological system playing a crucial role in modulating vessel tone and remodeling the blood flow and barrier function (Chand et al., 1981).

The properties of endothelial cells differ from tissue to tissue, which also are dependent on the location, function and structure of the specialized organ. The functions of various kinds of endothelial cells have been extensively studied by means of in vitro cultures. The structural and functional integrity of the endothelium is an essential requirement for its semiselective barrier properties between the blood stream and the underlying tissues. The maintenance of a continuous endothelial cell (EC) monolayer of tightly apposed cells is also central to preventing the vessel wall from platelet deposition and thrombus formation. Vascular endothelial cell monolayer functions as a barrier between the blood and interstitial compartments. A decrease in the barrier properties of vascular endothelium leads to tissue edema (Tiruppathi et al., 1992). Morphological studies in vitro and in vivo have shown the presence of tight junctions and gap junctions between adjacent endothelial cells (Schneeberger et al., 1984; Franke et al., 1988). Cell-cell adhesion structures have been extensively studied and specific molecules of both the calcium dependent and independent cell adhesion mechanisms have been identified (Cunningham, 1990; Takeichi, 1990). In spite of recognizing the importance and the molecular morphological organization of cell-cell junctions in the maintenance of endothelial functional properties, little is known about the physical properties of these sites (Lampugnani et al., 1992). For technical reasons, the electrophysiological investigation of these cells in situ has not been possible so far and therefore a number of questions concerning their basic function remain open.

The cytoskeleton of endothelial cells is an integrated three dimensional network of filaments and adhesion proteins in which mechanical forces at cell-cell regions are mechanically coupled to cell-matrix regions. These cell-cell and cell-matrix sites are mechanically coupled by an intervening series of filamentous cytoskeleton that can transfer local mechanical forces to distal sites. The cell-cell and cell-matrix adhesive sites provide a resistive barrier for the passage of macromolecules across a monolayer. The total transendothelial resistance provided by a monolayer comprises the cell-cell resistive component (denoted as R_{b}) and the cell-matrix resistive component (denoted by alpha) (Moy et al., 2002). These two resistances, along with the membrane capacitance (Cₘ), contribute to the total transcellular impedance. A lot of techniques like voltage clamp methods etc., have been developed to measure the permeability of tight junctions in epithelial cells, but these technologies have not been suitable to evaluate transendothelial resistance because of low transendothelial resistive values. Such low values of transendothelial resistance make its measurement very difficult and tedious. Electrical impedance measurements developed lately used fixed frequency to study endothelial cell barrier function (Geroski et al., 1992). Still further, another technique uses single frequency measurements to detect cellular micromotion in tissue culture (Giaever et al., 1984; Giaever et al., 1986; Giaever et al., 1989; Tiruppathi et al., 1992; Lo et al., 1993; Keese et al., 1994; Wegener et al., 2000; Noiri et al., 1998). Using a direct current does not give enough information about the cell barrier function, which can be obtained by using different current frequencies (AC).

An electrical method which was later used to study endothelial cells in real time so as to examine the mechanisms of alterations in the endothelial barrier function. Endothelial cytoskeleton properties were quantified by inoculating a monolayer of cultured endothelial cells grown on a small (10⁻⁴ cm²) evaporated gold electrode and measuring the changes in electrical impedance (Tiruppathi et al., 1992). In this system, referred to as electric cell-substrate impedance sensor (ECIS), the cells are cultured on a small gold electrode, and culture medium is used as the electrolyte (Giaever et al., 1984; Giaever et al., 1986; Giaever et al., 1989).

An approximately constant current source applies an alternating current signal of 1 micro ampere between this small electrode and a large counter electrode, while the voltage is monitored with a lock-in amplifier. A1 volt, 4000-Hz AC signal is supplied through a 1-M ohm resistor to approximate for the constant-current source. Voltage and phase data are stored and processed with a personal computer. The same computer controls the output of the amplifier and relay switches the measurement of different electrodes in the course of an experiment (Tiruppathi et al., 1992).

The barrier function is measured dynamically by determining the electrical impedance of a cell-covered electrode. The total impedance of the monolayer is composed of the impedance between the ventral surface of the cell and the electrode, the impedance between the cells, and the impedance of the cell membranes dominated by the membrane capacitance (Giaever et al., 1991). Membrane impedance is very large, and current flows along paths of least resistance, which includes flow under and between the cells and through transcellular conduction. The measured impedance primarily represents cell-cell adhesion and/or cell-matrix adhesion and membrane capacitance (Moy et al., 2000).

Initially, the ECIS system was designed for taking readings at a fixed frequency of 4000 Hz. By making impedance measurements over a range of frequencies, it was possible to numerically evaluate more than one barrier function parameter estimate. Using circular cell model geometry, Giaever et al., (1991) and Lo et al., (1995) estimated both paracellular and transcellular pathways from impedance measurements made over a range of frequencies in cultured fibroblasts and epithelial cells.

The ECIS system has certain limitations, for example, the microelectrode is highly sensitive to changes in frequency. The response obtained on the microelectrode is highly dependent on the frequency of the input current. At lower frequencies, the measured data does not fit well with the modeled solution parameters for cell-cell and cell-matrix adhesion and membrane capacitance calculated by the Levenberg Marquart Non linear Estimation (LM-NLS) method, and a significant error is associated with the solutions in lower frequencies. Additionally, at lower frequencies the ECIS electrode is dominated by a 1 nF capacitor that is in series with a resistor. Thus, the impedance is dominated by a frequency-dependent reactance, which decreases the sensitivity of the ECIS measurement at lower frequencies in detecting changes in biological membrane properties. At higher frequencies, the response of the microelectrode again becomes highly dependent on the parasitic capacitance of instrumentation components like the coaxial cables, which is a function of frequency. The ECIS system has both inherent low and high pass filtering properties. Hence, only a narrow range of frequencies are available which contain useful data and have steady impedance values.

Another limitation of the ECIS system is that the cell measurements are conducted on a rigid microelectrode instead of a more physiologically-relevant biomaterial that would better approximate the in vivo conditions. Cells are inoculated on a non-biological surface (metal) and there is no in vivo dynamics involved. Thus, the cytoskeletal properties of those cells might not be the same as they are in situ. Consequently, the cell membrane properties might not accurately reflect their physiological values. Also, the ECIS system only allows a response of cells to various biological interventions to be obtained only by challenging the apical surface of the cell. The basolateral surface cannot be challenged by providing any sort of exogenous stimulus, since that surface is not exposed on a microelectrode. Moreover, since there is a single channel for the current flow and voltage measurement, the system does not strictly remain a “constant current system.” Thus, there is a need to develop a system that can measure cell parameters in an in vivo condition.

### BRIEF SUMMARY OF THE INVENTION

The present invention is directed to a system and method which measures transcellular parameters in which cells are cultured in a monolayer on a biomaterial and parameters are measured that are indicative of in vivo values such as cell-cell and cell-matrix adhesion and cell membrane capacitance parameters.

One embodiment of the present invention comprises a method of measuring transcellular parameters in a cultured cell monolayer comprising: growing the cell monolayer on a biomaterial; affixing the biomaterial to a membrane; mounting the membrane in a chamber system; and measuring transcellular parameters selected from the group consisting of resistance, current, voltage, impedance, inductance and capacitance. These parameters can be measured at multiple current frequencies. More specifically, the transcellular parameters are indicators of cell adhesion, barrier function or transport.

In certain embodiments, the cell monolayer can be a monolayer of epithelial cells or endothelial cells. Epithelial and/or endothelial cells can be isolated from a variety of mammalian tissues, for example, but not limited to heart, skin, prostate, muscle, brain, prostate, breast, endometrium, lung, pancreas, small intestine, liver, testes, ovaries, cervix, colon, skin, stomach, esophagus, spleen, lymph node, or kidney. In addition to isolation of cells from tissues, numerous endothelial and/or epithelial cell lines and cultures are available for use, and they can be obtained through the American Type Culture Collection (ATCC).

Still further, the biomaterial may be a hydrogel. Exemplary hydrogels can include poly(lactide-co-glycolide) (PLG). PLG, poly (lactic acid), poly(ethylene oxide) (PEO), poly(vinyl alcohol) (PVA), poly(acrylic acid) (PAA), poly(propylene furmarate-co-ethylene glycol) (P(PF-co-EG)), polypeptides, agarose, alginate, chitosan, collagen, fibrin, gelatin, or hyaluronic acid (HA) , 2-hydroxyethyl methacrylate (HEMA)). More specifically, the hydrogel is a charged synthetic hydrogel that is a copolymer. More preferably, the copolymer comprises 2-methacryloxyethyl triethylammonium chloride (MAETA-Cl) or 2-hydroxyethyl methacrylate (HEMA).

In further embodiments, the membrane comprises one or more perforation. Perforations decrease background resistance in the circuit. In particular embodiments, the size of the perforations is in the range of about 100 to about 900 microns, more preferably, the perforations is about 400 microns.

Yet further, the chamber system may comprise at least one chamber and at least one electrode set. The chamber is a circulating chamber or a continuously perfused chamber. The chamber system comprises two electrode sets. The one electrode set is connected to a current clamp. The current clamp applies an AC current to the electrode set.

Another embodiment of the present invention is a cell culture insert used in a chamber system comprising: an insert having a tubular wall with open ends in which an internal passage extends from the opening of one end to the opening of the other end, and a support means located within the passage for supporting a perforated membrane that transverses the passage, the perforated membrane comprises a biomaterial affixed to the membrane, wherein the biomaterial supports a cell culture. The biomaterial is a charged synthetic hydrogel. In certain embodiments, the hydrogel can be poly(lactide-co-glycolide) (PLG). PLA, poly (lactic acid) poly(ethylene oxide) (PEO), poly(vinyl alcohol) (PVA), poly(acrylic acid) (PAA), poly(propylene furmarate-co-ethylene glycol) (P(PF-co-EG)), polypeptides, agarose, alginate, chitosan, collagen, fibrin, gelatin, hyaluronic acid (HA), or 2-hydroxyethyl methacrylate (HEMA).

Further embodiments of the present invention comprise a method of measuring barrier functions of cells comprising: growing a cell monolayer on the hydrogel of the cell culture insert; mounting the cell culture insert in a chamber system; and measuring barrier function by determining transcellular impedance.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows the diagrammatic view of an Ussing chamber, the current and the voltage electrodes and the flow system. The U-tubes contain the medium and the specimen is placed in the chamber. The gas supply (5% CO₂ and 95% O₂) is connected to the U-tubes. The current electrode supplies the constant current to the chamber and the voltage electrodes measures the voltage drop across the sample.

FIG. 2 illustrates the entire assembly that is placed in between the two chambers. The Teflon disk which acts as the supporting scaffold has 400 micrometer pore in its center. The hydrogel has the endothelial cells attached on its surface. The plastic disk and the rubber ring shown to the left of the monolayer are used to hold the hydrogel intact with the Teflon disk.

FIG. 3 shows the spindle shaped appearance of cultured endothelial cells on the first batch of gels.

FIG. 4 shows the desired morphology of endothelial cells obtained after keeping washing the gels in water for 4 weeks. The cells are inoculated on the hydrogel to confluency.

FIG. 5 shows the Frequency scan plot (Impedance Versus Frequency) of the only the media in the chamber. The response is flat until about 10 KHz.

FIG. 6 shows the Frequency scan Curves (Impedance Vs frequency) for all the Teflon pores. 400 μm pore size was selected amongst all of the above sizes. It provided the desired range of resistance and a flat response over the entire frequency spectrum.

FIG. 7 shows the Frequency scan curves for the finding the variation in impedance due to the hydrogel with changes in input current frequency. Readings were taken with and without the hydrogel on the 400 μm Teflon disk. The difference between the two displays the contribution of only the hydrogel towards aggregate impedance

FIG. 8 shows variation is resistive values over a frequency range from 0-100 KHz obtained on the Ussing chamber for the “cell covered” and the “naked hydrogel.”

FIG. 9 shows the normalized resistance values of the ratio ((R_{cc}/R_{nkd}) between the “cell covered” and the “naked hydrogel” across frequencies between 0-100 KHz.

FIG. 10 shows the impedance values provided by the monolayer isolated from the aggregate impedance. The plot was obtained from the difference of the cell-covered and the naked hydrogel, i.e., R_{cc}-R_{nkd}.

FIG. 11 shows the resistive values across the monolayer measured on the Ussing chamber between frequencies from 0-100 KHz

FIG. 12 shows the capacitive reactance measured on the Ussing chamber with the cells on the hydrogel, over a frequency range of 0-100 KHz

FIGS. 13A-13D show hydrogel modeling. FIG. 13A shows changes in normalized resistance by varying α and keeping the values of R_{b}, Cₘ and Rₘ fixed. FIG. 13B shows changes in normalized resistance by varying R_{b} and keeping the values of α, Cₘ and Rₘ fixed. FIG. 13C shows changes in normalized resistance by varying Cₘ and keeping the values R_{b}, α and Rₘ fixed. FIG. 13D shows changes in normalized resistance by varying Rₘ and keeping R_{b}, Cₘ and α fixed.

### DETAILED DESCRIPTION OF THE INVENTION

It is readily apparent to one skilled in the art that various embodiments and modifications can be made to the invention disclosed in this Application without departing from the scope and spirit of the invention.

I. Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

As used herein, the term “biomaterial” refers to any material or substance (other than a drug) or combination of substances that is intended to come into contact with biological systems. Biomaterials can be synthetic or natural in origin. Examples of biomaterials include, but are not limited to hydrogels.

As used herein, the term “hydrogel” refers to a biomaterial that consist of polymeric strands chemically crosslinked to form three-dimensional meshes, which are able to absorb water while maintaining structural integrity. Hydrogels can be natural polymers or synthetic polymers. Still further, a hydrogel can be a copolymer.

As used herein, the term “natural polymer” means any of a variety of long chain molecules that have repeating structural units that are derived from biologic (cellular) synthesis. Examples include collagens, gelatins (denatured collagen), fibrin, alginates, etc.

As used herein, the term “synthetic polymer” means any of a variety of long chain molecules that consist of a number of repeating structural unit that are derived in laboratory chemical synthesis.

As used herein, the term “endothelial cell” refers to cells that lines the blood vessels, and which the properties of the endothelial cells differ from tissue to tissue.

As used herein, the term “epithelial cell” refers to cells that cover or line all body parts both externally and internally. There are three types of epithelial cells, for example, squamous cells, cuboidal cells and columnar cells.

As used herein, the term “sample” refers to a biological sample, for example a tissue, or cell, more preferably a cell monolayer.

II. Present Invention

The present invention relates to a method and system to measure transendothelial impedance on a biomaterial surface (e.g., a hydrogel).

In certain embodiments, copolymer hydrogels are instituted which mimic the physiological milieu of endothelial cells in vivo. Copolymer hydrodgels are adhered to a membrane which is used in an Ussing chamber to measure transcellular impedance. These impedance readings are used for calculating the solution parameters of the cell by deriving a model equation analogous to the electric cell impedance sensing (ECIS) model.

Ussing chamber has been in use for many epithelial and tissue studies for measuring transmural resistances which involve probing the epithelium by direct current stimuli in order to determine the transepithelial resistance and conductance of intestinal tissues (Gitter et al., 1997). Such studies haven't been carried on the endothelium because of their low barrier function. The transendothelial impedance values are much lower than transepithelial resistive values. Thus, the present invention utilizes a biomaterial, e.g., hydrogels, in an Ussing chamber to eliminate the high inherent constrictive resistance such that it can be used to measure transendothelial impedance.

One of the advantages of measuring impedance on an Ussing chamber is that the alternating current can be applied at various frequencies. Hence considerable amount of information about the cell barrier function can be collected over a vast frequency spectrum. This cannot be achieved with direct current. Apart from these useful features, there are some added advantages of using the hyrdogel and the Ussing chamber system over the microelectrode. Unlike microelectrode, the monolayer inoculated on the hydrogel may be challenged with an exogenous stimuli on both apical and basolateral surfaces. Thus, the properties and response of the basolateral surface can also be revealed on an Ussing chamber. Another advantage of using the Ussing chamber for impedance measurements is that it truly provides a constant current to the cells. The microelectrode has a single path for the current and voltage measurements. Hence the current supplied to the cells doesn't strictly remain a “constant current.” An Ussing chamber has separate current and voltage electrodes. The current electrodes supply a constant current to the chamber and the voltage electrodes are placed very near to the specimen. This configuration ensures that a constant current is maintained between the voltage electrodes.

III. Biomaterials

In certain embodiments of the present invention a cell monolayer is grown on a biomaterial. Biomaterials are any material or substance (other than a drug) or combination of substances that is intended to come into contact with biological systems. In the present invention, the biomaterial must be capable of supporting cell growth. Exemplary biomaterials can include hydrogels, polyethylene, polyacetal, terylene, ceramics, polydimethylsiloxane and polyhydroxybutyrate.

In one embodiment, the biomaterial is a hydrodgel. Hydrogels consist of natural or synthetic polymeric strands chemically crosslinked to form three-dimensional meshes, which are able to absorb large amounts of water while maintaining their structural integrity. Synthetic polymers include, but are not limited to poly(lactide-co-glycolide) (PLG). PLA, poly (lactic acid) poly(ethylene oxide) (PEO), poly(vinyl alcohol) (PVA), poly(acrylic acid) (PAA), poly(propylene furmarate-co-ethylene glycol) (P(PF-co-EG)), hyaluronic acid (HA), or 2-hydroxyethyl methacrylate (HEMA). Natural polymers include, but are not limited to, polypeptides, agarose, alginate, chitosan, collagen, fibrin, or gelatin. Commercially available hydrogels include but are not limited to PuraMatrix™ Peptide Hydrogel (3DM Inc., Cambridge, Mass.).

Hydrogels can also be copolymers. Copolymer hydrogels represent important biomaterials in the field of biotechnology and medicine and have been used in many medical and tissue engineering applications (Pedley et al., 1980; Dai et al., 2000). Hydrogels undergo different swelling transitions based on their chemical compositions and ambient conditions, such as temperature, pH, and ionic strength. The ability of hydrogels to retain water largely determines the transport properties within the membranes; water can be exchanged with low molecular weight molecules, such as ions and metabolites, from the surrounding fluid. This enables the maintenance of a chemical balance within the copolymer, similar to that of biological tissue. During polymerization, integrating different amounts and kinds of functional groups within the networks can incorporate charge in them. Moreover, adding a charged moiety affects the amount of swelling of the hydrogel. Swelling is an imperative behaviour of an hydrogels which influences its transport properties (Friedl et al., 2000; Folkman et al., 1978; Wachem et al., 1987; Bergethon et al., 1989; Faris et al., 1983; McFarland et al., 2000; Smetana et al., 1990; Wichterle et al., 1960).

Cell migration and morphology is controlled by selective adhesive interactions with the copolymer surface, which are greatly influenced by surface chemistry (Wachem et al., 1987; Bergethon et al., 1989; Faris et al., 1983; Smetana et al., 1990; Iio et al., 1994; Bermeister et al., 1996; Drumheller et al., 1994). Investigators have studied, specifically, the influence of positively- and negatively-charged functional groups on cellular growth and have shown that incorporation of charged (positive or negative) moieties encourages cell growth. (Wachem et al., 1987; Iio et al., 1994; Jacobson et al., 1982).

Studies on effect of charged hydrogel with varying amount of charged and neutral monomers on cellular adhesion and proliferation are known for various cell types. The optimal charges of copolymer polyelectrolyte hydrogels for supporting proper attachment, growth and proliferation of porcine pulmonary arterial endothelial cells (PPAEC) have been reported to be +160 and +200 (Kari Haxinasto, masters' thesis). The charged monomer (basic moiety) in such hydrogels is Methacryloxyethyltrimethyl ammonium chloride (MAETA-Cl) and 2-hydroxyethyl methacrylate (HEMA) is the neutral monomer. Thus, in certain embodiments of the present invention, the hydrogel is a copolymer of MAETA-Cl and HEMA. Other copolymer hydrogels that can be used in the present invention are further described in U.S. Patent Application No. 20040096505, which is incorporated herein in its entirety.

Additional examples of hydrogels that can be used in the present invention are described in further detail in U.S. Pat. No. 6,171,610, which is incorporated by reference in its entirety, and include, but are not limited to: (1) temperature dependent hydrogels [e.g., PLURONICS™ (BASF-Wyandotte), such as polyoxyethylene-polyoxypropylene F-108, F-68, and F-127, poly (N-isopropylacrylamide), and N-isopropylacrylamide copolymers]; (2) ionic hydrogels [e.g., alginates or chitosan]; (3) light sensitive hydrogels [e.g., polyethylene oxide block copolymers, polyethylene glycol polylactic acid copolymers with acrylate end groups, and 10 K polyethylene glycol-glycolide copolymer capped by an acrylate at both ends]; and (4) pH dependent hydrogels [e.g., TETRONICS™ (BASF-Wyandotte) polyoxyethylene-polyoxypropylene polymers of ethylene diamine, poly(diethyl aminoethyl methacrylate-g-ethylene glycol), and poly(2-hydroxymethyl methacrylate)].

IV. Chamber Systems

The chamber system or apparatus in accordance with the invention comprises, as one of its elements, an insert having a tubular wall with opposite ends, an opening in each end, an internal passage extending from the opening at one end to the opening at the other end, and means, at a location within the passage, for supporting a membrane barrier such as animal tissue or a monolayer of cells grown on a porous substrate.

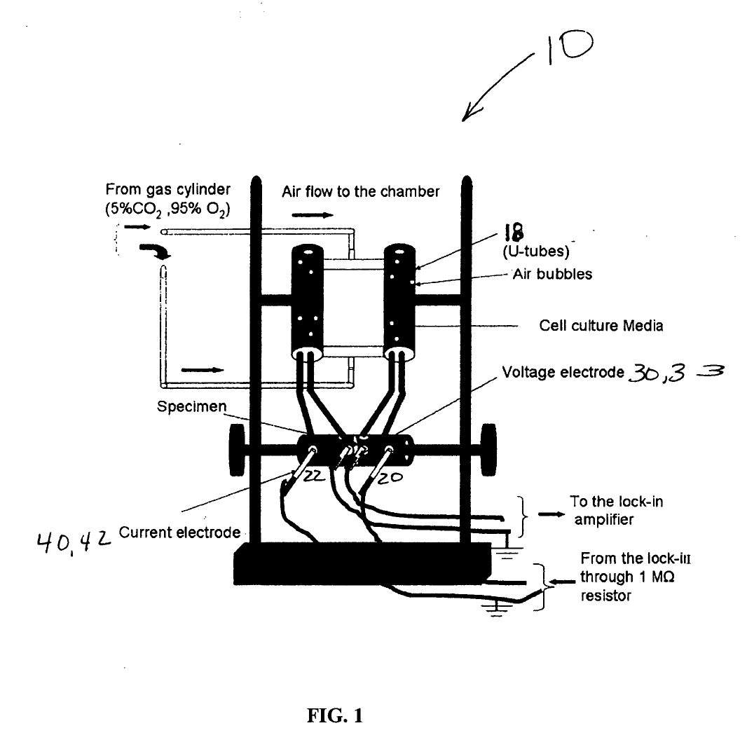

An exemplary type of chamber system that can be used in the present invention is an Ussing-type chamber system, further described in U.S. Pat. No. 5,738,826 and PCT Publication WO96\02627 each of which is incorporated herein by reference in its entirety. The Ussing chamber consists of the chambers and the electrical circuitry. A lot of technical variations for both of these subunits have been developed. An exemplary type of chamber used in the present invention is shown in FIG. 1. Commercially available Ussing-type chamber systems are available from World Precision Instrument, Inc.; Warner Instruments, and SDR Clinical Technology.

A. Chambers

Two types of chambers can be used, which include a circulating chamber and a continuously perfused chamber. Each of the chambers provide solutions bathing the two sides of the sample. The solutions are delivered to the chamber from the reservoirs mounted above the chamber via polyethylene tubes into the inside of the chamber. Valves may be used to regulate the flow rate, which is otherwise dependent on the diameter of the tubes and the hydrostatic pressure. Temperature is adjusted by means of the water jacket heating system (Martin J Hug).

1. Circulating Chambers

The circulating chamber consists of a U-shaped tubing system usually made of glass that is filled with the cell culture medium or any other solution of interest, as shown in FIG. 1. The tubing is heated by a water jacket which encases the tubing. The water is maintained at 37° C. by a heated circulating bath. The tubing is gassed with air or different gases such as CO₂, O₂, N₂ etc. For example, growth of a monolayer of cells may require a mixture of 5% CO₂ and 95% O₂. Other mixtures of gases may also but utilized depending upon the samples are used. The gassing serves two functions: 1) it oxygenates the liquid contents and 2) it stirs the liquid to ensure complete convection (“bubble lift”).

The lumen of the tube is connected to the chambers. The U-shaped tube secures an equal hydrostatic pressure on both sides of the chamber and thus avoids damage caused by bending of the sample.

Turning to FIG. 1, the Ussing chamber 10 is provided with a solution circulation and temperature regulating device 18, for circulating solution within the Ussing chamber. The Ussing chamber 10 comprises two halves 20 and 22, which are essentially identical to each other and are in face-to-face relationship with each other. Each chamber half is formed from a synthetic resin such as polymethylmethacrylate, known commercially as Plexiglas or Lucite, or polytetrafluoroethylene, known commercially as Teflon. The chamber halves comprise an interface in which the sample can be placed.

2. Continuously Perfused Chambers

Continuously perfused chambers are designed such that the two half chambers minimize the hydrostatic pressure, and thus prevent damage to the sample during perfusion.

B. Electrodes

In addition to the chamber, the Ussing system comprises electrodes, more particularly, at least one electrode set. The electrode of the first pair is located within the hollow interior space of one of the two bodies and the other electrode of the first pair is located within the hollow interior space of the other of the two bodies.

A second pair of electrodes may also be provided. One electrode of the second pair extends through the hollow interior space of one of the two bodies and has a tip located in close proximity to the porous substrate, within the internal passage of the cell culture insert, on one side thereof. The other electrode of the second pair extends through the hollow interior space of the other of the two bodies and has a tip located in close proximity to the porous substrate on the other side thereof. At least one of the electrodes of the second pair extends through the opening in one of the ends of the tubular wall of the cell culture insert.

The electrodes can be either voltage type electrodes or current type electrodes. Various types of electrodes can be used, for example, but not limited to KCl filled glass column, agar-bridge, and calomel electrode. In certain embodiments, the present invention comprises both current and voltage electrodes.

Further examples of electrodes that can be used in the present invention include reference electrodes (electrodes used as a ground) for example, saturated calomel (SCE), calomel Hg/Hg₂Cl₂, silver-silver chloride Ag/AgCl, mercury-mercury oxide Hg/HgO, mercury-mercurous sulfate Hg/Hg₂SO₄, copper-copper sulfate Cu/CUSO₄, and non-aqueous Ag. In addition to reference or ground electrodes, working electrodes that are used to measure the electrical property of the sample can also be used. Examples of working electrodes include, but are not limited to platinum, gold, silver, glassy carbon, nickel and palladium. Examples of implantable electrode materials include, but are not limited to noble metals (e.g., gold, iridium, platinum, rhodium, ruthenium, palladium, mercury, silver), passive metals (e.g., copper, tin, indium, gallium, tantalum, niobium, chromium, zirconium, aluminium, hafnium, titanium, beryllium, tungsten), metals and depolarisers (e.g., Ag—AgCl, Pt—Pt“black”), alloys (e.g., gold-platinum-rhodium, platinum-iridium, platinum-rhodium) and carbons.

Thus, in certain embodiments, at least one electrode pair or electrode set is provide, more preferably two sets of electrodes or two electrode pairs are provided. For example, one electrode pair is a current electrode, while the second electrode pair is a voltage electrode. More particularly, a set of voltage and current Ag/AgCl electrodes can be used. Each electrode can be in a 3 M KCl Agarose Bridge. The agar gel solution was made by dissolving 3.0% agarose by weight into the 3.0 M KCl solution at 80° C., which is pipetted into the polyethylene casings of the electrodes with the extrusion of all noticeable bubbles. The electrodes can be immediately immersed in the agarose solution that cooled and set around them. For later use, the electrodes can be stored in 3.0 M KCl solution to avoid depletion of ions.

As shown in FIG. 1, a constant current source can be provided by using a resistor (1 M-ohm) in the current path from the lock-in amplifier. The current electrodes or first set of electrodes 40 and 42, which are located at the extreme ends of the two half chambers 20 and 22, are used for delivering current to the sample which is placed in between chambers 20 and 22. This type of configuration is essentially a current clamp circuit, in which the current frequency can be altered over a range of about 0 Hz to about 100 KHz by using the lock-in amplifier. Voltage electrodes or the second set of electrodes 30 and 32 are inserted into two slots in the chambers 20 and 22 near the sample, which are used to measure the potential drop across the sample. In-phase voltage drop can be due to the resistive component in the current path and the out of phase potential drop can be due to the capacitive component. These measured voltage readings are used to calculate the impedance across the sample.

C. Cell Culture Insert

In certain embodiments of the present invention, an insert is utilized in the chamber for cell growth, in which the insert comprises a scaffold or membrane support and a biomaterial affixed to the membrane.

The scaffold or membrane are cut into circular disk, which will fit exactly inside the chamber. The membrane may be various thickness to produce the desired result. Preferably, a biocompatible membrane of a device of the present invention is about 0.15 inches.

In certain embodiments, the membrane support is comprised of polytetrafluoroethylene (Telfon™). Other materials that can be used for the membrane providing the material comprises the desired insulation and elasticity requirements, for example, but not limited to glass (e.g., quartz glass, lead glass or borosilicate glass), silicon, silicon dioxide on silicon, silicon-on-insulator (SOI) wafer, sapphire, plastics, and polymers. Some preferred polymers are polyimide (e.g., Kapton, polyimide film supplied by DuPont), polystyrene, polycarbonate, polyvinyl chloride, polyester, polyethylene terephthalate (PET), polypropylene and urea resin. It is preferable, that any durable nonstick electrical insulation material may be used.

In order to decrease the background resistance, the membrane supports comprise at least one hole or perforation to decrease the background resistance in the circuit. The perforation or hole is typically placed in the center of the membrane, however, the hole may be place in other areas of the membrane. The size of the perforations is in the range of about 100 μm to about 900 μm, more preferably in the range of about 100 μm to about 200 μm; about 200 μm to about 250 μm; about 250 μm to about 300 μm; about 300 μm to about 350 μm; about 350 μm to about 400 μm; about 400 μm to about 450 μm; about 450 μm to about 500 μm; about 500 μm to about 600 μm; about 600 μm to about 700 μm; about 700 μm to about 800 μm; about 800 μm to about 900 μm; or any range there between. In specific embodiments, the size of the perforations is about 400 μm.

Once the membrane support is constructed, then a biomaterial is selected to be affixed to the membrane support. Any type of biomaterial can be used in the present invention as long as the material can support a monolayer of cultured cells. Thus, any of the biomaterials, more specifically hydrogels, which are described above in detail and incorporated by reference into this section can be affixed to the membrane support.

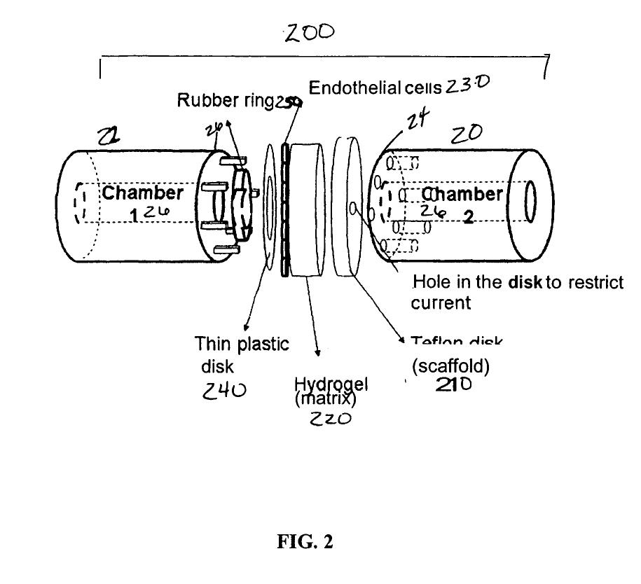

FIG. 2 illustrates an exemplary cell insert that can be used in the chamber system. Each half chamber 20 and 22 has a hollow interior space or internal passage 26, which extends from the opening of one end to the opening of the other end, which is open to the face of the chamber half at interface 24, and 26, respectfully. An insert for receiving a cell culture insert 200 is assembled and placed between interface 24 and 26.

Insert 200 comprises a membrane 210 (e.g., Teflon disk) in which a biomaterial 220 (e.g., hydrogel) is affixed to the membrane 210. A cell monolayer 230 can be grown on the biomaterial 220. A plastic disk 240 and rubber ring 250 are used to hold the biomaterial 220 intact with the membrane 210.

V. Use of a Modified Ussing Chamber to Measure Transcellular Parameters

Certain aspects of the present invention comprise measuring transcellular parameters of a cultured cell monolayer. The cells are grown on a biomaterial, in the biomaterial is then affixed to a membrane. This membrane is mounted in a chamber system, for example an ussing Chamber system, as described above and transcellular parameters are measured such as resistance, current, voltage, impedance, inductance and capacitance. These measurements can occur at multiple current frequencies.

Cells that can be cultured on the biomaterial of the present invention include but are not limited to epithelial cell and/or endothelial cells. Epithelial and/or endothelial cells can be isolated using well known and used techniques in the art from a variety of mammalian tissues. Tissues that can be used for isolation of cells include, for example, but not limited to heart, skin, prostate, muscle, brain, prostate, breast, endometrium, lung, pancreas, small intestine, liver, testes, ovaries, cervix, colon, skin, stomach, esophagus, spleen, lymph node, or kidney. In addition to isolation of cells from tissues, numerous endothelial and/or epithelial cell lines and cultures are available for use, and they can be obtained through the American Type Culture Collection (ATCC), which is an organization that serves as an archive for living cultures and genetic materials.

The cell monolayer is adherent to the biomaterial. As used herein, the term adherent refers to the attachment of cells to the biomaterial such that the cells are capable of growing to confluency on the biomaterial. Thus, preferably at least 5%, 10%, 20% to about 50% of the cells adhere to the biomaterial within 12 to 24 hours. The properties of the biomaterial allow for growth of a confluent monolayer of cells on the biomaterial prior to usage in the chamber system. A confluent monolayer of cells refers to having at least 80% to about 100% of the biomaterial covered by a monolayer of cells.

Once the cell inset is placed in the chamber, the Ussing chamber is assembled and used to measure impedance. The current electrodes at the two extreme ends of the two chambers are used for delivering current to the specimen placed in the center of the chamber. A 1 volt constant current source is provided by using a 1 M-ohm resistor in the current path from the lock-in amplifier such that the system is a current clamp circuit. This enables the current frequency to be altered over a range of 0 Hz-100 KHz using the lock-in amplifier. The voltage electrodes are connected very close to the chamber cross-section and are placed on each side of the chamber to measure the potential drop across the cross-section (due to the inserted sample or cell monolayer between the two chambers).

The in-phase and out of phase components of the drop are separately displayed on the lock-in amplifier front panel display. The in-phase voltage drop is due to the resistive component in the current path and the out of phase potential drop is due to the capacitive component. The measured voltage readings are used to calculate the impedance across the sample.

Based upon the impedance data obtained from the Ussing chamber, the data are used to determine other cellular parameters, such as cell-cell adhesion parameter (denoted by R_{b}), cell-matrix adhesion parameter (denoted by alpha) and membrane capacitance (denoted by Cₘ) using the below equation:

$$\frac{1}{{Z}_{c}} = \frac{1}{{Z}_{n}} ( \frac{{Z}_{n}}{{Z}_{n} + {Z}_{m}} + \frac{\frac{{Z}_{m}}{{Z}_{n} + {Z}_{m}}}{\frac{ⅈγ {r}_{c} {I}_{0} ( γ {r}_{c} )}{2 {I}_{1} ( γ {r}_{c} )} + 2 {R}_{b} ( \frac{1}{{Z}_{n}} + \frac{1}{{Z}_{m}} )} )$$

$$Where , γ {r}_{c} = {r}_{c} \sqrt{\frac{ρ}{h} ( \frac{1}{{Z}_{n}} + \frac{1}{{Z}_{m}} )} = α \sqrt{\frac{1}{{Z}_{n}} + \frac{1}{{Z}_{m}}}$$

In this model, the total impedance across a cell-covered matrix is composed of the impedance of the cell monolayer (related to alpha), the impedance between cells (indicated by R_{b}), the transcellular impedance (Zₘ), and the impedance of a naked matrix (Zₙ). For these calculations, the cells are regarded as circular disks and Zₘ is inversely related to membrane capacitance (Cₘ). Alpha (α) is defined as α=R_{c}\sqrt{ρ/h} where R_{c} is the cell radius, ρ is the resistivity of the medium, and h is the average separation distance between the cell and the underlying matrix (Moy et al., 2002).

### VI. EXAMPLES

The following examples are included to demonstrate preferred embodiments, more particularly methods and procedures, of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cell Isolation and Preparation

PPAEC were isolated from porcine pulmonary arteries obtained from a local abattoir. After resecting several inches of a pulmonary artery, each end was clamped using a hemostat and the artery was quickly dipped in 70% ethanol and then rinsed thoroughly with Medium 199. The arteries were then transferred back to the laboratory in Medium 199 containing penicillin (100 μg/mL) and streptomycin (100 μg/mL) (penicillin-streptomycin solution GibcoBRL Cat. No. 15140-122). All cells were cultured in Medium 199 supplemented with 20% heat-inactivated FBS, basal medium Eagle vitamins and amino acids, and glutamine (2 mM). Endothelial cells were cultured using Medium 199 penicillin and streptomycin supplemented with L-glutamine (GibcoBRL Cat. No. 25030-081), BME amino acids (Sigma Chemical Co, Cat. No. B-6766), BME vitamin (Sigma Chemical Co., B-6891), and fetal calf serum (Hyclone Inc.). Under a laminar flow hood, having been wiped with 70% ethanol 5 minutes prior, 2 mL of 50:50 conditioned medium were added to a 35 mm tissue culture dish. Each artery was longitudinally dissected open with sterile scissors and was held open using sterile tweezers. The lumen was washed with conditioned Medium199 solution and the intimal layer of endothelial cells was gently scrapped from the luminal surface using a sterile scalpel. The scraping motion was done in one direction, never over the same area twice, since this might dislodge smooth muscle cells. The cells were transferred to a 35 mm Petri dish, containing 2 mL of 50:50 conditioned Medium 199, by lightly tapping the scalpel blade on the Petri dish surface. These cultures were then transferred to a tissue culture incubator with humidification, set at 5.0% CO₂ and 37° C.

After 4 hours the medium from the 35 mm culture dishes was removed and replaced with 2 ml of fresh conditioned medium. The culture was maintained until the endothelial cells achieved confluency after approximately 7 to 10 days. The culture was also checked visually to ensure that the cobblestone morphology was maintained without signs of smooth muscle contamination. The endothelial cells were then passaged into a 60 mm tissue culture dish using a 0.05% trypsin-EDTA solution (Gibco, Inc). Approximately 1 week later when the endothelial cells in the 60 mm dish had reached confluence, each dish was passaged into one 100 mm tissue culture dish. The endothelial cells were then passaged once a week at a ratio of 1:4. Cell lines were not passaged beyond passage ten. Porcine pulmonary artery endothelial cells between passages four and ten were used for this study (Kari Haxinasto, Masters' thesis).

### Example 2

### Hydrogel Preparation

The copolymer hydrogels were fabricated according to techniques established by Kari Haxinasto and Anthony English (Kari Haxinasto, (Masters' thesis).

Briefly, two molar stock solutions of each of the monomers (MAETAC and HEMA) were prepared with a 20.2 mM concentration of Ethylene glycol dimethacrylate (EGDMA), which acts as a cross-linker. An aqueous solvent with 40% ethylene glycol (EG) was also used for each stock solution. The polymerization reactions were initiated with 1.76 mM Ammonium Persulfate (APS) and accelerated using 7.6 mM sodium metabisulfite (SMBS). The solutions were then mixed in two different proportions to produce the desired copolymer hydrogels with acidic or basic monomer concentrations of +160 and +200 mM. The chemical concentrations used to prepare these different charged HEMA and MAETA-Cl, copolymers are shown in table 1.

TABLE 1
Chemical composition of each stock solution
used during hydrogel fabrication

| | Formula Weight/ | Concen- | |
|---|---|---|---|
| Chemical | density/purity % | tration | Mass/Volume |
| HEMA | 130.14 g/mol/ | 2.0 M | 64.215 mL/250 mL |
| | 1.034 g/ml/98% | | |
| MAETAC | 207.7 g/mol/70% | 2.0 M | 14.836 mL/25 mL |
| EGDMA | 198.22 g/mol | 20.2 mM | 40 uL/10 mL |
| EG | | | 4 mL/10 mL |
| APS | 228.2 | 1.76 mM | 100 μL 4 wt %/10 mL |
| SMBS | 190.1 | 7.6 mM | 100 μL 15 wt %/10 mL |

2-hydroxyethyl methacrylate (HEMA) is the neutral monomer, Methacryloxyethyltrimethyl ammonium chloride (MAETA-Cl) is the charged monomer, Ethyleneglycol dimethacrylate (EGDMA) is the crosslinker, Ammonium persulfate (APS) is the initiator and Sodium metabisulfite (SMBS) is the activator for the polymerization process.

Prior to polymerization, the hydrogel samples were degassed under a vacuum so that there is no interference of oxygen during the polymerization process. After the stock solutions were degassed, the appropriate proportions of ionic stock solutions were added to aliquots of the neutral stock solutions to produce samples of basic pregellation solutions with concentrations of 160, and 200 mM. To each 10 mL sample a 100 μL aliquot of 15 wt % SMBS was added to accelerate the polymerization reaction.

Hydrogel membranes were formed by casting several milliliters of the pregellation solution between two glass plates separated by a thin Teflon film spacer. Small metal binder clips were used to form a tight seal between the two glass plates. All samples were allowed to polymerize at 25° C. for 24 hours.

After polymerization was complete, the hydrogels were removed from between the glass plates and placed in distilled deionized water where they were washed continuously with daily water changes for two weeks. In this way, residual non-polymerized chemicals were removed from the gels. This water bath was then systematically replaced with logarithmically spaced concentrations of NaCl dissolved in distilled deionized water, beginning with a 10-6 M NaCl solution and ending with a 1M NaCl solution. The gels were washed in each NaCl solution for the appropriate amount of time to allow for their equilibration (Kari Haxinasto, Masters' thesis).

### Example 3

### Cell Inoculation on the Hydrogel

Once the hydrogels were completely detoxified by continuous washing in distilled water and equilibrating them with NaCl solution, they were cut into small circular disks. The diameter of these disks was equal to the diameter of the chamber so that they could be easily mounted. These circular disks of gel were cut using a number 11 borer (11 mm diameter borer from Small parts Inc). The gels were later soaked in 150 mM NaCl solutions and kept in tissue culture bottles for storage. In order to sterilize the gels, they were autoclaved for half an hour before inoculating cells on them.

### Example 4

### Platting Density for the PPAEC

Porcine pulmonary arterial endothelial cells (PPAEC) were used to carry the out the impedance measurements.

The cells were cultured in the lab and cells no older than passage 10 were used for any of the experiments. Individual gels were put in separate wells of a P-12 delta dish and the cells were platted on top of the gels. After inoculating the cells several times on different hydrogels, it was realized that a count of around 700,000 cells/gel/well was optimal for obtaining a confluent monolayer of cells on the gels.

### Example 5

### Cell Morphology and Attachment Issues

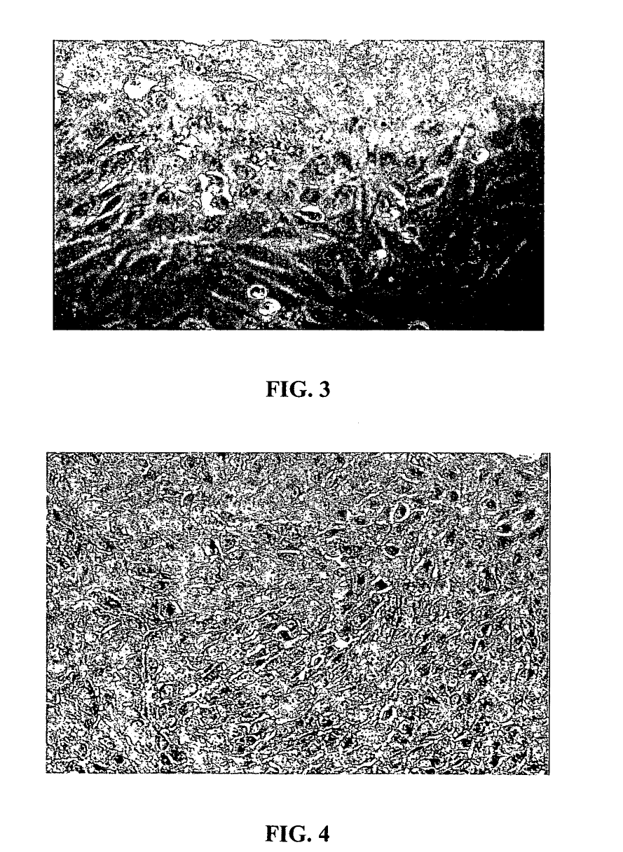

The morphology of the cells did not look proper on the first batch of gels that were fabricated. The cells were observed a day after they were inoculated on the gels. Instead of the expected cobblestone appearance of endothelial cells, they had a spindle appearance (FIG. 3)

A new set of gels were fabricated and were washed with distilled water for 4 weeks instead of 2 weeks with daily water change. The new batch of gel was cut into circular disks and inoculated with cells. The cells had the perfect cobblestone appearance on the new gels (FIG. 4). Thus it was realized that washing the gels for 2 week might not be enough to detoxify them. Henceforth, all the batches of gels were washed for 4 weeks in distilled water followed by 2 weeks of washing with varying concentration of NaCl solutions.

### Example 6

### Measurements on the Ussing chamber

The Ussing chamber was used for all experiments involving impedance measurements. The current electrodes at the two extreme ends of the two chambers were used for delivering current to the specimen placed in the center of the chamber. A 1 volt constant current source was provided by using a 1 M-ohm resistor in the current path from the lock-in amplifier. Hence the system was essentially a current clamp circuit. The current frequency was altered over a range of 0 Hz-100 KHz using the lock-in amplifier. The agar encased voltage electrodes connected very close to the chamber cross-section and were placed on each side of the chamber to measure the potential drop across the cross-section (due to the inserted sample between the two chambers). Thus, the voltage electrodes essentially measured the potential drop across the specimen. The in-phase and out of phase components of the drop were separately displayed on the lock-in amplifier front panel display. The in-phase voltage drop was due to the resistive component in the current path and the out of phase potential drop was due to the capacitive component. The measured voltage readings were used to calculate the impedance across the sample. The current and voltage electrodes were connected in a manner such that Chamber 1 (Cl) was grounded as shown in the (FIG. 1). Before and after each experiment, the agar bridges encasing the electrodes were fully saturated with KCl by immersing them in a 3.0 M KCl solution. The bridges were also tested before and after each experiment in to ensure that negligible drift had occurred between the electrodes because of ion diffusion. In every case, the electric potential between electrodes was negligible (<0.3). An electrode potential offset reading taken was (0.0-0.1 mV) which had negligibly contribution to the overall potential.

After the electrodes were tested for drift, the Ussing chamber was dismantled and the specimens were placed between the two chambers. The two voltage electrodes (FIG. 2) were inserted in the two slots very near to the specimen, on either side of the chambers. The chambers were then reconnected and the flow columns were connected to the Ussing chamber. The current electrodes were also connected to the Ussing chamber chamber and the medium or the solution of interest was poured slowly from the top of both the columns. 5% CO₂ and 95% O₂ gas flowed through the Ussing chamber at the rate of 3-4 psi. The in and out of phase voltage readings were taken from the lock in amplifier and separately recorded. The current frequencies were changed manually by rotating the frequency knob on the lock-in front panel. After each set of experiment, the Ussing chamber was washed several times with distilled water to ensure that any residual fluid or residue was washed away.

### Example 7

### Measuring the Impedance Contributed by the Media

The circuit impedance was measured to assess the contribution of different components. Voltage readings were taken from the Ussing chamber which consisted of only serum contained medium. The experimental conditions were kept such that 5% CO₂ and 95% O₂ supply was set at 3-5 psi and the water heating bath was maintained at 37° C. to keep the medium warm. The impedance readings were recorded on the lock-in amplifier (Stanford Instruments Inc) over a frequency range of 0 Hz to 100 KHz.

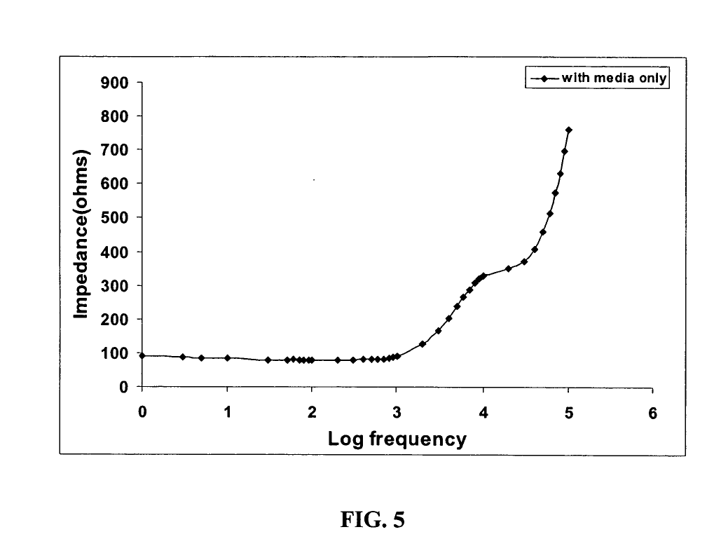

The relationship between frequency and resistance is shown in FIG. 5. It was observed that the impedance due to medium increased exponentially with increase in the current frequency. The impedance values due to the medium were less than 400 ohms up to the frequency of 10 KHz. The impedance increases sharply after 10 KHz and reaches values up to 1000 ohms. Hence, the contribution of the medium was estimated and recorded.

### Example 8

### Teflon Disks as Scaffolds for the Hydrogels

The prepared hydrogels were very fragile and delicate when manipulated in the Ussing chamber without any kind of support. Moreover the area through which the current passes in an ordinary chamber is very large. Since the resistance of the bulk tissue culture medium is in series with the impedance of the cells, it would dominate the measurement if the area through which the current passes is large. In contrast, having a very small path for the current would increase the faradic impedance of the current path. This would make the background constrictive resistance much more than the cellular contribution to the total impedance measured. Hence an optimal pore size was calculated which would restrict the path of current density and would provide an optimal resistance without overshadowing the contribution of the cell monolayer.

For the purpose of providing a support in the Ussing chamber, Teflon was selected as the material for the hydrogel scaffold. It provided the desired insulation and elasticity required for our purpose. Teflon sheets were cut in circular disks of the size that would just fit exactly inside the chamber. The sheets were 0.015 inch thick. Pores of varying sizes were drilled in the center of these disks from 100 μm, 200 μm, 250 μm, 300 μm, 400 μm, 500 μm, 600 μm, 700 μm, 800 μm and 900 μm. These disks were sequentially placed between the two Ussing chambers and voltage drops across them was measured over a frequency range of 0 Hz-100 KHz. For all sets of readings, the experimental conditions were kept constant. The U-tubes and the chambers were filled with medium and the gas supply was passed through the chamber during the entire course of experiment. The medium was kept warm at a temperature of 37° C.

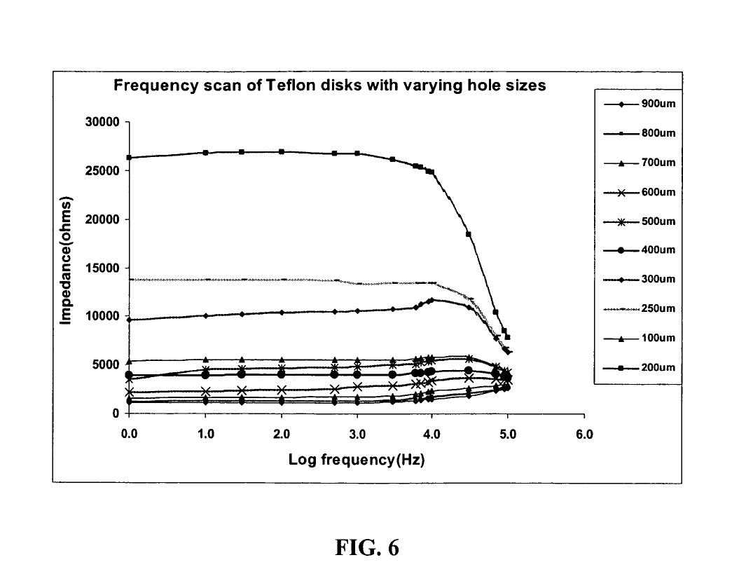

The impedance values for all the pore sizes of the Teflon disks were plotted between frequencies 0-100 KHz. This Frequency scan plot (Impedance vs Frequency) was used to select the best pore size which could be used for collecting data with the hydrogel and the cells. The purpose was to find out the optimal pore size for the Teflon disk which could be used for optimizing faradic resistance from a microscopic pore but at the same time did not increase the background resistance. All measures were taken to keep the conditions consistent for all sets of readings. FIG. 6 shows the plot of impedance observed by the Teflon disks over a frequency range of 0-100 KHz.

The resistance provided by the Teflon disks decreased linearly with increase in pore size. This behavior wasn't demonstrated by the 100 μm disk. After analyzing the results, 400 μm was considered as the best size for the pore due to two reasons. First, the resistance measured with a Teflon disk with a pore size of 400 μm was 3000-4000 Ohms. The results obtained from the ECIS electrodes have shown that the impedance contribution due to PPAEC is nearly 4000-6000 Ohms. These data demonstrated that the constriction resistance offered by the 400 μm Teflon disk was less than the faradaic resistance offered by monolayer. Hence, the cellular contribution could be easily detected and separated from the aggregate impedance using a 400 μm pore size. Second, the resistance values for the 400 μm disk over the entire range of frequencies from 0 Hz to 100 KHz were very steady and constant. Such a response was not observed with many other pore sizes (FIG. 6).

### Example 9

### Contribution of the Hydrogel

After finding the optimal pore size for the Teflon scaffold, the contribution of the hydrogel to the resultant impedance readings was determined. The hydrogel was placed on top of the Teflon disk such that it covered the entire area including the pore through which the current would pass. Thus, the hydrogel acted as an impeding barrier in the current path and hence had a contribution to the resultant impedance measurement. Since Teflon is a non-stick material, the hydrogel had to be adhered to it using a different approach. A very thin plastic disk with a small pore inside it (much bigger than the Teflon pore through which the current passes), was placed on the hydrogel after it was kept on top of the Teflon disk. A rubber ring was then placed on the plastic disk which held the plastic disk and the underlying hydrogel in its place. This entire assembly (FIG. 2) was placed in between the chamber. The disk and the ring made the hydrogel stay coupled to the Teflon disk. Since they did not lie in the current path, they had no contribution to the measured impedance. The entire assembly was ensured to be electrically insulated with no leakage of current from the chamber. Voltage readings under similar conditions were taken with the hydrogel on the chosen Teflon disk. The gas was supplied throughout the course of the experiment and the medium with serum was kept warm at 37° C. Voltage readings at frequencies between 0 Hz to 100 KHz were recorded.

FIG. 7 shows the impedance values as a function of frequency. It was observed that the hydrogel exhibited negligible impedance values, which ranged between 100-300 ohms. Thus, compared to the resistance of the Teflon, the hydrogel resistance was insignificant.

### Example 10

### Cell Covered Reading

To obtain impedance offered by the cells, the entire assembly comprising of the cells on the hydrogels, the Teflon scaffold, the plastic disk and the rubber ring, were placed inside the Ussing chamber. The plastic disk and a rubber ring were used to hold the hydrogel intact with the Teflon, as previously described. The media (with serum) along with the mixture of 5% CO₂ and 95% O₂, continuously perfused the chamber. The entire assembly was maintained at a temperature of 37° C. with the water heating jacket. The in-phase and out-of phase voltage drop across the monolayer and the hydrogel were measured by the lock-in amplifier. This voltage drop was measured over current frequencies between 0 Hz to 100 KHz. Thus, the entire range of impedance for the frequency spectrum was obtained. These readings were referred as the “Cell covered” readings.

### Example 11

### Cell-Free Readings

After measuring the cell covered impedance readings from the hydrogels, the cells were detached, which was achieved by first flushing out the media and then incubating cells with 0.05% trypsin-EDTA in the chamber from the U-tubes. The trypsin was incubated for several minutes after which it was flushed out. The chamber was again filled with the serum medium. All other conditions were kept similar to the earlier experiments and voltage readings from 0 Hz up to 100 KHz were measured and recorded. The obtained readings constituted the “naked-hydrogel” readings. Alternatively, the “cell-free reading can be obtained by performing a measurement of a new cell-free hydrogel. The variance for cell-free hydrogels was within ˜500 ohms—impedance levels much less than the difference between cell-covered and cell-free hydrogel.

### Example 12

### Impedance with the Cells on the Hydrogels

The goal behind evaluating all the aforementioned measurements was to measure the impedance offered by the cellular monolayer, which reflected a measurement of the endothelial barrier function.

The assembly comprising of the Teflon disk, the cells attached to the hydrogel, the plastic disk and the rubber ring as mentioned previously, were mounted in the Ussing chamber and voltage measurements were taken at frequencies between 0-100 KHz.

After measuring voltages for the “Cell covered” hydrogel, the cells were subsequently trypsinized and removed from the hydrogel, or a new cell-free hydrogel was mounted into the chamber to take readings for the cell free or the “naked” hydrogel. Voltage measurements were taken between the same frequencies from 0-100 KHz.

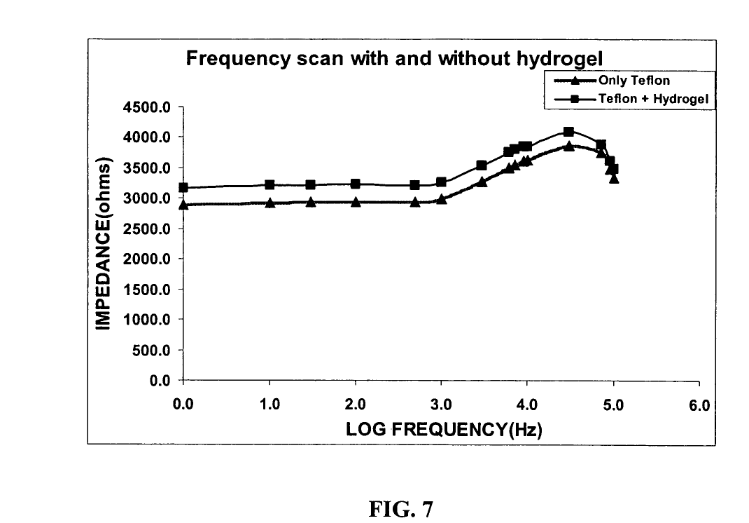

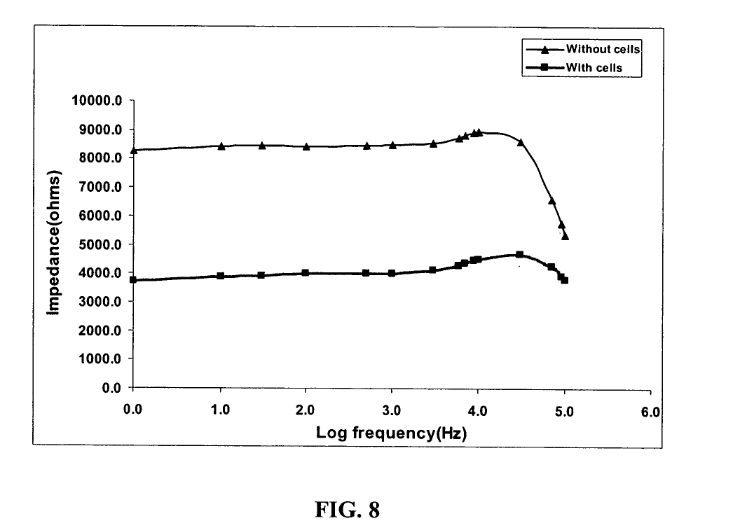

FIG. 8 shows a combined plot for both the “cell-covered” and “naked hydrogel”. As evident from the graph, the cellular monolayer contributes nearly 4500 ohms to the total impedance. The impedance values remain nearly constant in the low and medium frequencies and decline drastically after 10 KHz. The difference between the cell covered and naked hydrogels is very distinct and the hence the cellular contribution can be easily separated.

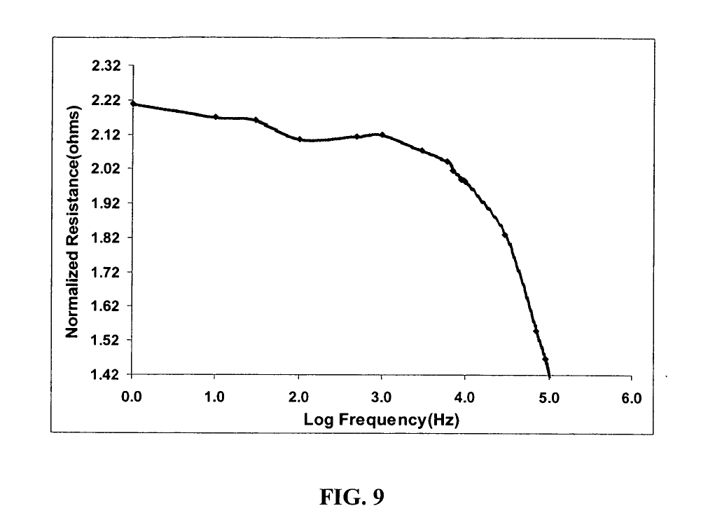

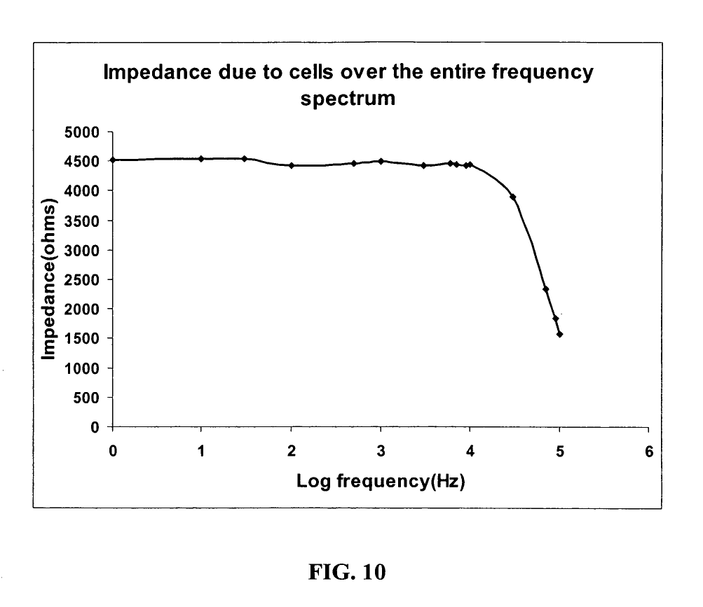

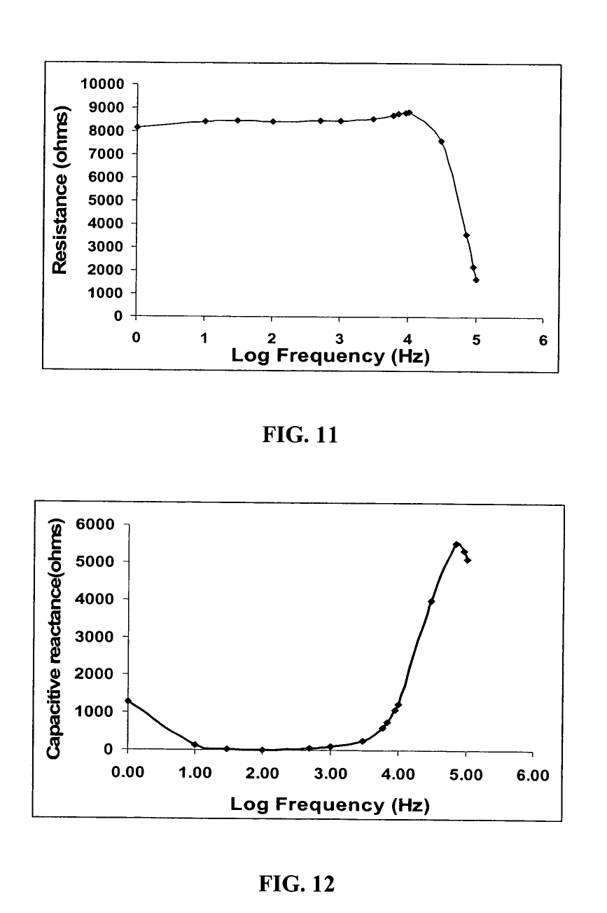

FIG. 10 shows the plot of impedance Vs frequency for the cellular monolayer. FIG. 11 shows the resistance values across the frequency spectrum from 0-100 KHz and FIG. 12 shows the capacitive reactance values over the same frequencies range between 0-100 KHz.

### Example 13

### Hydrogel Modeling

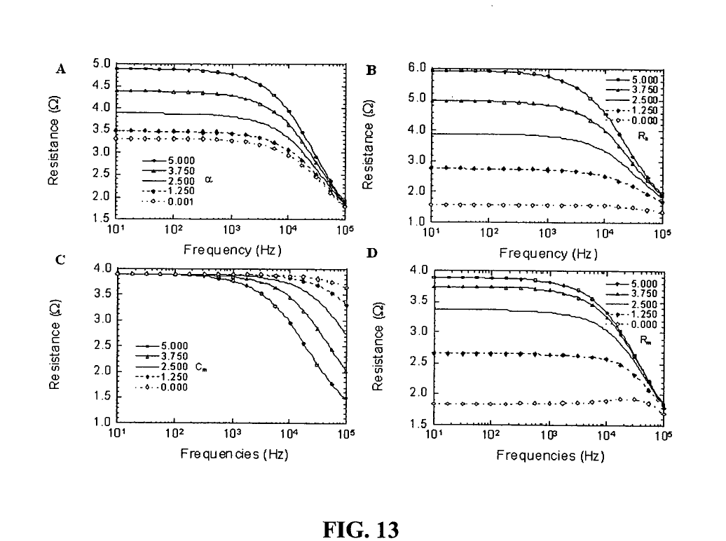

The impedance data obtained for the cells on the Ussing chamber was modeled. The cell membrane parameters such as the cell-cell adhesion parameter (R_{b}), cell matrix adhesion parameters (α) and the membrane capacitance (Cₘ) were calculated and plotted. Rₘ is the resistance offered by the cell membrane due to ion conductance. The hydrogel model takes into consideration this extra resistive parameter, which is in parallel to Cₘ and collectively represents the model of transmembrane impedance (Zₘ). Using a representative parameter set obtained from the actual experiment, the impact of systematic changes in α, R_{b}, Cₘ, and Rₘ for the normalized curves were determined. FIG. 13 shows the changes in normalized resistance (ratio of cell-covered to cell free hydrogel) corresponding to changes in the four parameters α, R, Cₘ, and Rₘ from a Ag/AgCl electrode on an ussing chamber The naked impedance is assumed to be constant 3-4 kΩ with a negligible reactive component.

FIG. 13A shows the variation due to the parameter α by fixing R_{b}, Cₘ and Rₘ at constant values. Similarly, FIG. 13B) shows the normalized resistance values generated by varying R_{b} and keeping α, Rₘ and Cₘ fixed. FIG. 13C) shows the normalized resistances values generated by varying Cₘ and keeping α, Rₘ and R_{b} fixed. FIG. 13D) shows the normalized resistances values generated by varying Rₘ and keeping α, R_{b} and Cₘ fixed.

The fixed parameter values were as follows: α=2.5 Ω^{0.5} cm, R_{b}=2.5 Ωcm², Cₘ=2.5 μFCₘ² and Rₘ=10.0 Ωcm². It was observed that by keeping all the other parameters fixed, increase in α value increases the total normalized resistance. Such a behavior is observed until current exceeded a frequency of 10 KHz, after which the normalized resistance becomes less sensitive to changes in the α values. Similar observations were recorded for R_{b} and Rₘ values also, where in, increasing their values increased the normalized resistance until the frequency exceeded 10 KHz. For frequencies greater than 10 kHz, the normalized resistance was less sensitive to αR_{b} and Rₘ. Thus, the data showed that the solution parameters have their unique frequency contribution only on the resistance values below 10 KHz. In contrast, varying Cₘ values had more profound effect on resistance above a frequency of 10 KHz. There is a greater drop in normalized resistance observed with increasing Cₘ=values at high frequencies. Thus, these observations suggest that the model predicts that the resistance offered by the monolayer on the hydrogel below a current frequency of 10 KHz is a function of α, R_{b} and Rₘ values but is solely a function of Cₘ values at higher frequencies. Another very important conclusion obtained from the observations is that the measurements obtained from the Ussing chamber were sensitive to changes in all the membrane parameters. Hence, the system was successful in measuring all the membrane parameters, which are presently obtained from the microelectrode.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Aplin, A. E., et al., 1998. Pharmacol Rev 50, no. 2:197.
Ben-Ze'ev, A. 1983. J Cell Biol 97, no. 3:858.
Bergethon, P. R., et al., 1989. J Cell Sci 92, no. Pt 1: 111.
Burmeister, J. S., et al., 1996. J Biomed Mater Res 30, no. 1:13.
Carson, M., et al., 1989. Am. J. physiolo.257 L259-L264.
Chand, N., and B. M. Altura, 1981. Science 213: 1376-1379)
Chen, W. T., et al., 1985. J Cell Biol 100, no. 4:1103.
Tirruppathi, C., et al., 1992. Proc Nat'l Acad Sci USA 89: 7919-7923, 1992.
Dai, W. S., Barbari, T. A. 2000. Biomaterials 21:1363.
Drumheller, P. D., and J. A. Hubbell. 1994. Anal Biochem 222, no. 2:380
Fadel, M. P., et al., 1999. J Biol Chem 274, no. 21:15085.
Faris, B., et al., 1983. Exp Cell Res 143, no. 1:15.
Folkman, J., and A. Moscona. 1978. Nature 273, no. 5661:345.
Franke, R. R., et al., 1988. J. Biol. Chem. 263, 2119-2122.
Friedl, P., and E. B. Brocker. 2000. Cell Mol Life Sci 57, no. 1:41.
Geroski, D. H. and A. Hadley, 1992 Current Eye Research 11:61-72.
Giaever I and Keese C R. 1984. Proc Nat'l Acad Sci USA 81: 3761-3764.
Giaever I and Keese C R. 1986. IEEE Trans Biomed Eng 33: 242-247.
Giaever, I. and C. R. Keese, 1989. Physica D38:128-133.
Giaever I and Keese C R. 1991. Proc Nat'l Acad Sci USA 88: 7896-7900.
Gitter H. A., et al., June 1997, journal of biochemical and biophysical methods)
Griffloen, A. W., and G. Molema. 2000. Pharmacol Rev 52, no. 2:237.
Hug Martin, Universitat Munster, Munster, Germany
Iio, K., et al., J Biomed Mater Res 28, no. 4:459.
Izzard, C. S., and L. R. Lochner. 1976. J Cell Sci 21, no. 1:129.
Jacobson, B. S. a. U.S.R. 1982. Tissue and Cell 14, no. 1:69.
Kari Haxinasto, Masters thesis, December 2001.
Lampugnani M G, et al., J Cell Biol. 1992 Sep;1 18(6):1511-22.
Liu, J. C.; Candidacy Proposal, California Institute of Technology, 2002.
Lo C, Keese C, and Giaever I. Biophys J 69: 2800-2807, 1995.
Lo, C.-M., et al., Experimental Cell Research 204:102-109, 1993.
McFarland, C. D., et al., 2000. J Biomed Mater Res 49, no. 2:200.
Milos Pekny, et al., Glia. 1998 Apr;22(4):390-400.
Moy A B, et al., J Clin Invest 97: 1020-1027, 1996.
Moy A, et al., Am J Physiol Lung Cell Mol Physiol 274: L1024-L1029, 1998.
Moy A, et al., Am J Physiol Lung Cell Mol Physiol 278: L888-L898, 2000.
Moy A. B, et al., Am J Physiol Lung Cell Mol Physiol 278: L888-L898, 2000.
Moy A. B, et al., Am J Physiol Heart Circ Physiol. 2002 Jan;282(1):H21-9.
Noiri, E., et al., Am. J. Physiol. 274:C236-C244, 1998.
Olschewski Andrea, et al., Am J Respir Cell Mol Biol. 2001 September;25(3):285-90
Panos Kouklis, et al., Circ Res. 2004 Feb. 6;94(2):159-66.
Pedley, D. G., et al., 1980. British Polymer Journal 12:99.)
Persechini A and Hartshorne D. Science 213: 1383-1385, 1981.
Pitelka D, et al., J Cell Biol 96: 613-624, 1983.
Schmid-Schonbein G, et al., J. Biomech Eng 117: 171-178, 1995.
Schneeberger E E, and Lynch R T; 1984, Circ Res 55: 723-733.
Shasby D M and Shasby S. J Appl Physiol 60: 71-79, 1986.
Shasby S S, et al., Cir res 51, 657-661, 1982
Sheldon R, et al., Am J Physiol Lung Cell Mol Physiol 265: L606-L612,1993.
Silvia Nistri, et al., Biol. Proced. Online 2002;4(1): 32-37.
Smetana, K., Jr., et al., J Biomed Mater Res 24, no. 4:463.
Takeichi M; Annu Rev Biochem. 1990;59:237-52.
Tiruppathi C, et al., Proc Nat'l Acad Sci USA 89: 7919-7923, 1992.
Van Wachem, P. B., et al., 1987. Biomaterials 8, no. 5:323.
Wegener, J., et al. Experimental Cell Research 259:158-166, 2000.
Wichterle, O. a. L., D. 1960. Nature 185:117.
Yamato, M., M. et al., 1999. J Biomed Mater Res 44, no. 1:44.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of measuring transcellular parameters in a cultured adherent cell monolayer comprising:
growing the cell monolayer on a biomaterial;
affixing the biomaterial to a membrane;
mounting the membrane in a chamber system; and
measuring transcellular parameters selected from the group consisting of resistance, current, voltage, impedance, inductance and capacitance.

2. The method of claim 1, wherein the cell monolayer is a monolayer of epithelial cells or endothelial cells.

3. The method of claim 1, wherein the transcellular parameters are indicators of cell adhesion, barrier function or transport.

4. The method of claim 1, wherein the biomaterial is a hydrogel.

5. The method of claim 4, wherein the hydrogel is poly(lactide-co-glycolide) (PLG). PLG, poly (lactic acid), poly(ethylene oxide) (PEO), poly(vinyl alcohol) (PVA), poly(acrylic acid) (PAA), poly(propylene furmarate-co-ethylene glycol) (P(PF-co-EG)), polypeptides, agarose, alginate, chitosan, collagen, fibrin, gelatin, and hyaluronic acid (HA), or 2-hydroxyethyl methacrylate (HEMA).)

6. The method of claim 4, wherein the hydrogel is a charged synthetic hydrogel.

7. The method of claim 6, wherein the hydrogel is a copolyer comprising 2-methacrylooxyethyl triethylammonium chloride (MAETA-Cl) and 2-hydroxyethyl methacrylate (HEMA).

8. The method of claim 1, wherein the transcellular parameters are measured at multiple current frequencies.

9. The method of claim 1, wherein the membrane comprises perforations.

10. The method of claim 9, wherein the size of the perforations is in the range of about 100 to about 900 microns

11. The method of claim 10, wherein the size of the perforations is about 400 microns.

12. The method of claim 10, wherein the perforations decrease background resistance in the circuit.

13. The method of claim 1, wherein the chamber system comprises at least one chamber and at least one electrode set.

14. The method of claim 13, wherein the chamber is a circulating chamber or a continuously perfused chamber.

15. The method of claim 13, wherein the chamber system comprises two electrode sets.

16. The method of claim 15, wherein the one electrode set is connected to a current clamp.

17. The method of claim 16, wherein the current clamp applies an AC current to the electrode set.

18. A cell culture insert used in a chamber system comprising:
an insert having a tubular wall with open ends in which an internal passage extends from the opening of one end to the opening of the other end, and
a support means located within the passage for supporting a perforated membrane that transverses the passage, the perforated membrane comprises a biomaterial affixed to the membrane, wherein the biomaterial supports a cell culture.

19. The cell culture insert of claim 18, wherein the biomaterial is a charged synthetic hydrogel.

20. The cell culture insert of claim 18, wherein the hydrogel is copolyer comprising 2-methacrylooxyethyl triethylammonium chloride (MAETA-Cl) and 2-hydroxyethyl methacrylate (HEMA).

21. The cell culture insert of claim 18, wherein the size of the perforations in the membrane is about 100 to about 900 microns.

22. The cell culture insert of claim 20, wherein the size of the perforations is about 400 microns.

23. The cell culture insert of claim 18, wherein the cell culture is a monolayer of endothelial cells or epithelial cells.

24. A method of measuring barrier functions of cells comprising:
growing a cell monolayer on the biomaterial of the cell culture insert of claim 16;
mounting the cell culture insert in a chamber system; and
measuring barrier function by determining transcellular impedance.

25. The method of claim 25, wherein the cell monolayer is a monolayer of endothelial cells or epithelial cells.

26. The method of claim 25, wherein the biomaterial is a charged synthetic hydrogel.

27. The method of claim 26, wherein the hydrogel is a copolymer comprising 2-methacryloxyethyl triethylammonium chloride (MAETA-Cl) and 2-hydroxyethyl methacrylate (HEMA).
